(19) United States
(12) Patent Application Publication
(10) Pub. No.: US 2016/0198657 A1
(43) Pub. Date: Jul. 14, 2016

(54) LATE EMBRYO DEVELOPMENT AND MATURATION AT COLDER TEMPERATURE

(71) Applicant: WEYERHAEUSER NR COMPANY, FEDERAL WAY, WA (US)

(72) Inventor: Pramod K. Gupta, Federal Way, WA (US)

(73) Assignee: WEYERHAEUSER NR COMPANY, FEDERAL WAY, WA (US)

(21) Appl. No.: 14/989,511

(22) Filed: Jan. 6, 2016

Related U.S. Application Data
(60) Provisional application No. 62/101,306, filed on Jan. 8, 2015.

(51) Int. Cl. A01H 4/00

(52) U.S. Cl. CPC A01H 4/005

(57) ABSTRACT

A method for developing conifer cotyledonary somatic embryos is described. The method includes culturing conifer pre-cotyledonary somatic embryos in, or on, a development medium at a temperature from about 20° C. to about 22° C. for a first incubation period sufficient in length for at least a portion of the pre-cotyledonary somatic embryos to develop into cotyledonary somatic embryos, and culturing the cotyledonary somatic embryos in, or on, the development medium at a temperature from about 10° C. to about 15° C. for a second incubation period sufficient in length for at least a portion of the cotyledonary somatic embryos to reach anatomical maturity.

### CROSS-REFERENCE TO RELATED APPLICATION

This application is entitled to and claims the benefit of priority under 35 U.S.C. §119 from U.S. Provisional Patent Application Ser. No. 62/101,306 filed Jan. 8, 2015, and titled LATE EMBRYO DEVELOPMENT AND MATURATION AT COLDER TEMPERATURE, the contents of which are incorporated herein by reference.

### BACKGROUND

The demand for coniferous trees, such as pines and firs, to make wood products continues to increase. One proposed solution to the problem of providing an adequate supply of coniferous trees is to identify individual coniferous trees that possess desirable characteristics, such as a rapid rate of growth, and to produce numerous, genetically identical, clones of the superior trees by somatic cloning.

Somatic cloning is the process of creating genetically identical trees from tree somatic tissue. Tree somatic tissue is tree tissue other than the male and female gametes. In one approach to somatic cloning, tree somatic tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development of cotyledonary somatic embryos which can, for example, be placed within artificial seeds and sown in the soil where they germinate to yield conifer seedlings. The seedlings can be transplanted to a growth site for subsequent growth and eventual harvesting to yield lumber, or wood-derived products. The cotyledonary somatic embryos can also be germinated in a germination medium, and thereafter transferred to soil for further growth.

There is a continuing need to improve the efficiency of somatic cloning of conifer embryos in order to increase production of cotyledonary somatic embryos that are capable of germinating to yield conifer trees. It is preferable that the conifer somatic embryos formed in vitro are physically and physiologically similar, or identical, to conifer zygotic embryos formed in vivo in conifer seeds. Methods that address this need with respect to conifers are described herein.

### SUMMARY

In one aspect, a method for developing conifer cotyledonary somatic embryos is provided. The method comprises the steps of (a) culturing conifer pre-cotyledonary somatic embryos in, or on, a development medium at a temperature from about 20° C. to about 22° C. for a first incubation period sufficient in length for at least a portion of the pre-cotyledonary somatic embryos to develop into cotyledonary somatic embryos; and (b) culturing the cotyledonary somatic embryos developed in step (a) in, or on, the development medium at a temperature from about 10° C. to about 15° C. for a second incubation period sufficient in length for at least a portion of the cotyledonary somatic embryos to reach anatomical maturity.

In one embodiment, the conifer pre-cotyledonary somatic embryos consist essentially of Loblolly pine pre-cotyledonary somatic embryos. In one embodiment, the conifer pre-cotyledonary somatic embryos consist essentially of Douglas-fir pre-cotyledonary somatic embryos.

In one embodiment, the development medium comprises about 10% PEG to about 16% PEG. In one embodiment, the development medium comprises about 10% PEG to about 12% PEG.

In one embodiment, the first incubation period is from about 5 weeks to about 8 weeks. In one embodiment, the first incubation period is from about 6 weeks to about 7 weeks. In one embodiment, the second incubation period is from about 4 weeks to about 7 weeks. In one embodiment, the second incubation period is from about 5 weeks to about 6 weeks. In one embodiment, the first and second incubation periods together total about 12 weeks.

In one embodiment, in step (b) the cotyledonary somatic embryos are cultured in, or on, the development medium at a temperature of 12° C.

In one aspect, a method for developing conifer cotyledonary somatic embryos is provided. The method comprises the steps of (a) culturing conifer somatic cells in, or on, an induction medium to provide embryogenic cells; (b) culturing the embryogenic cells provided in step (a) in, or on, a maintenance medium to provide pre-cotyledonary somatic embryos; (c) culturing the pre-cotyledonary somatic embryos provided in step (b) in, or on, a development medium at a temperature from about 20° C. to about 22° C. for a first incubation period sufficient in length for at least a portion of the pre-cotyledonary somatic embryos to develop into cotyledonary somatic embryos; and (d) culturing the cotyledonary somatic embryos developed in step (c) in, or on, the development medium at a temperature from about 10° C. to about 15° C. for a second incubation period sufficient in length for at least a portion of the cotyledonary somatic embryos to reach anatomical maturity.

In one embodiment, the conifer pre-cotyledonary somatic embryos consist essentially of Loblolly pine pre-cotyledonary somatic embryos. In one embodiment, the conifer pre-cotyledonary somatic embryos consist essentially of Douglas-fir pre-cotyledonary somatic embryos.

In one embodiment, the development medium comprises about 10% PEG to about 16% PEG. In one embodiment, the development medium comprises about 10% PEG to about 12% PEG.

In one embodiment, the first incubation period is from about 5 weeks to about 8 weeks. In one embodiment, the second incubation period is from about 4 weeks to about 7 weeks. In one embodiment, the first and second incubation periods together total about 12 weeks.

In one embodiment, in step (d) the cotyledonary somatic embryos are cultured in, or on, the development medium at a temperature of 12° C.

### DESCRIPTION OF THE DRAWINGS

The foregoing aspects and many of the attendant advantages of the described methods will become more readily appreciated as the same become better understood by reference to the following detailed description, when taken in conjunction with the accompanying drawings, wherein:

FIG. 1 shows the germination percentage for category 1 germinants for three genotypes used in Example 1;

FIG. 2 shows the germination percentage for category 1 germinants for four genotypes used in Example 1;

FIG. 3 shows the LS-means and confidence intervals for the data shown in FIG. 2;

FIG. 4 shows the germination percentage for category 1 and category 2 germinants for three genotypes used in Example 1;

FIG. 5 shows the germination percentage for category 1 and category 2 germinants for four genotypes used in Example 1;

FIG. 6 shows the LS-means and confidence intervals for the data shown in FIG. 5;

FIG. 7 shows the percent increase of category 1 germinants in Treatment 8 as compared to the control treatment;

FIG. 8 shows the percent increase of category 1 and category 2 germinants in Treatment 8 as compared to the control treatment;

FIG. 9 shows the root length for category 1 germinants for three genotypes used in Example 1;

FIG. 10 shows the germination percentage for category 1 germinants for genotypes used in Example 1;

FIG. 11 shows the germination percentage for category 1 germinants for each of the three genotypes used in Example 2;

FIG. 12 shows the LS-means and confidence intervals for the data shown in FIG. 12;

FIG. 13 shows the root length for each category 1 germinant by genotype;

FIG. 14 shows the LS-means and confidence intervals for the data shown in FIG. 13;

FIG. 15 shows the hypocotyl length for each category 1 germinant by genotype;

FIG. 16 shows the LS-means and confidence intervals for the data shown in FIG. 15;

FIG. 17 shows the epicotyl length for each category 1 germinant by genotype;

FIG. 18 shows the LS-means and confidence intervals for the data shown in FIG. 17; and

FIG. 19 shows the germination percentage for category 1 germinants for each of the four genotypes used in Example 3.

### DETAILED DESCRIPTION

Unless specifically defined herein, all terms used herein have the same meaning as they would to one skilled in the art of the described subject matter.

In accordance with the methods described herein, it has been unexpectedly discovered that culturing immature, pre-cotyledonary conifer somatic embryos in, or on, a development medium at a temperature from about 20° C. to about 22° C. for a first incubation period sufficient in length for at least a portion of the pre-cotyledonary embryos to develop into cotyledonary somatic embryos (typically a time period of about 5-8 weeks), followed by culturing the cotyledonary somatic embryos in, or on, the development medium at a temperature from about 10° C. to about 15° C. for a second incubation period sufficient in length for at least a portion of the cotyledonary somatic embryos to reach anatomical maturity (typically a time period of about 4-7 weeks), produced embryos with increased germination frequency and vigor compared to embryos that were cultured in, or on, a development medium at a temperature from about 20° C. to about 21° C. for a period of about 12 weeks.

As used herein, the term “development stage” refers to the period during somatic cloning during which histogenesis and growth of tissues and organs occurs in an immature embryo to reach a full-sized mature embryo capable of germination into a plant.

As used herein, the term “somatic embryo” refers to a plant embryo that developed in vitro from a plant cell, or tissue, that is not a zygote.

As used herein, the term “immature embryo” refers to an embryo that is not yet capable of germination into a plant, and includes embryos in early stage development (i.e., pre-cotyledonary embryos), and mid-stage development (i.e., embryos with cotyledons or hypocotyls that are not yet fully developed).

As used herein, the term “pre-cotyledonary embryo” refers to an embryo that does not yet have cotyledons.

As used herein, the term “cotyledonary embryo” refers to an embryo with a well-defined, elongated bipolar structure with latent meristematic centers having one or more clearly visible cotyledonary primordia at one end and a latent radicle at the opposite end.

As used herein, the term “anatomical maturity” refers to an embryo that possesses developed cotyledons and hypocotyl.

As used herein, the term “normal germinant” denotes the presence of all expected parts of a plant at time of evaluation. The expected parts of a plant can include a radicle, a hypocotyl, one or more cotyledon(s), and an epicotyl. In the case of gymnosperms, a normal germinant is characterized by the radicle having a length greater than 3 mm and no visibly discernible malformations compared to the appearance of embryos germinated from natural seed.

As used herein, the term “radicle” refers to the part of a plant embryo that develops into the primary root of the resulting plant.

As used herein, the term “hypocotyl” refers to the portion of a plant embryo or seedling located below the cotyledons but above the radicle.

As used herein, the term “epicotyl” refers to the portion of the seedling stem that is above the cotyledons.

As used herein, the term “embryonal suspensor mass” or “ESM” refers to a cell mass plated onto the surface of nutrient medium contained either in a semi-solid gel or as a liquid in a porous matrix capable of providing physical support, and left to grow for a period up to three months. During the three-month incubation time, somatic embryos grow from microscopic precursor cell groups into visible early-stage embryos and eventually to anatomically mature embryos. The structure of the ESM after several weeks of incubation typically consists of a proliferated mat with a few embryos sitting in direct contact with media, but most embryos have formed on the top or side of the still proliferating cell mass.

As used herein, the term “stratification” refers to subjecting embryos to a cold treatment (e.g., 0° C. to 10° C.) prior to germination. Stratification (moist chilling) is a treatment used for overcoming germination resistance in the seeds of many temperate plant species (Taylor & Waring, Plant, Cell, And Environment 2:165-171, 1979).

As used herein, the term “category 1 germinant” refers to a normal germinant of very good quality on visual inspection. Category 1 germinants have a root at least 1 cm in length with epicotyls at least 1 cm in length.

As used herein, the term “category 2 germinant” refers to a normal germinant of good quality on visual inspection. Category 2 germinants have a root about 0.2 cm in length and an epicotyl about 0.5 cm in length.

As used herein, the term “category 1+2 germinants” refers to the total number of category 1 and category 2 germinants.

Unless stated otherwise, all concentration values that are expressed as percentages are weight per volume percentages.

In one aspect, a method for developing conifer cotyledonary somatic embryos is provided. The method comprises (a) culturing conifer pre-cotyledonary somatic embryos in, or on, a development medium at a temperature from about 20° C. to about 22° C. for a first incubation period sufficient in length for at least a portion of the pre-cotyledonary somatic embryos to develop into cotyledonary somatic embryos; and (b) culturing the cotyledonary somatic embryos developed in step (a) in, or on, the development medium at a temperature from about 10° C. to about 15° C. for a second incubation period sufficient in length for at least a portion of the cotyledonary somatic embryos to reach anatomical maturity.

The methods described herein can be used to produce mature cotyledonary somatic embryos from any conifer, such as members of the genus Pinus, such as Loblolly pine (Pinus taedti) and Radiata pine. By way of example, Douglas-fir embryos can be produced by the described methods.

Thus, in one embodiment, the conifer pre-cotyledonary somatic embryos consist essentially of Loblolly pine pre-cotyledonary somatic embryos. In another embodiment, the conifer pre-cotyledonary somatic embryos consist essentially of Douglas-fir pre-cotyledonary somatic embryos.

A population of mature conifer somatic embryos produced according to the described methods was surprisingly found to have a greater efficiency of germinating into conifer trees than a population of conifer somatic embryos produced according to an otherwise identical control method where conifer pre-cotyledonary somatic embryos remain in, or on, development medium at a temperature from 20° C. to 22° C. for a length of time sufficient for a portion of the pre-cotyledonary somatic embryos to reach anatomical maturity, usually a period of about 12 weeks.

Immature conifer somatic embryos, such as, for example, pre-cotyledonary conifer somatic embryos, can be prepared from conifer somatic cells, such as cells obtained from conifer embryos. For example, cells from conifer embryos can be induced by hormones to form embryonal suspensor cell masses (ESMs) that can be treated in accordance with the described methods to yield mature conifer somatic embryos. ESMs can be prepared, for example, from pre-cotyledonary embryos removed from seed. The seeds can be surface sterilized before removing the pre-cotyledonary embryos, which are then cultured on, or in, an induction medium that permits formation of ESMs which include early state embryos in the process of multiplication by budding and cleavage. ESMs are typically cultured in a maintenance medium to form pre-cotyledonary somatic embryos. Non-limiting examples of ESM culture conditions and suitable induction and maintenance media are further described below.

Induction.

Conifer somatic cells are cultured in, or on, an induction medium to yield embryogenic cells. Embryogenic cells are cells that are capable of producing one or more cotyledonary conifer somatic embryos and include, for example, conifer embryonal suspensor masses. The induction medium typically includes inorganic salts and organic nutrient materials. The osmolality of the induction medium is typically about 120 to 200 mM/kg. The induction medium typically includes growth hormones. Examples of hormones that can be included in the induction medium are auxins (e.g., 2,4-dichlorophenoxyacetic acid (2,4-D)) and cytokinins (e.g., 6-benzylaminopurine (BAP)). Auxins can be used, for example, at a concentration of from 1 mg/L to 200 mg/L. Cytokinins can be used, for example, at a concentration of from 0.1 mg/L to 10 mg/L.

The induction medium can contain an absorbent composition, particularly when very high levels of growth hormones are used. The absorbent composition can be any composition that is not toxic to the embryogenic cells at the concentrations used, and also capable of absorbing growth-promoting hormones and toxic compounds produced by plant cells during embryo development and present in the medium. Non-limiting examples of useful absorbent compositions include activated charcoal, soluble poly(vinyl-pyrrolidone), insoluble poly(vinyl-pyrrolidone), activated alumina, and silica gel. The absorbent composition can be present in an amount, for example, of from about 0.1 g/L to about 5 g/L. The induction medium is typically solid and can be solidified by inclusion of a gelling agent. An example of an induction medium useful in the practice of the described methods is medium BM1 as described in Table 1.

Conifer somatic cells are typically cultured in, or on, an induction medium for a period of from 3 weeks to 10 weeks, such as from 6 weeks to 8 weeks, at a temperature of from 10° C. to 30° C., such as from 15° C. to 25° C., or such as from 20° C. to 22° C.

Maintenance.

The maintenance medium can be a solid medium or it can be a liquid medium which is agitated to promote growth and multiplication of the embryogenic tissue. The osmolality of the maintenance medium is typically higher than the osmolality of the induction medium, for example, in the range of 120 to 250 mM/kg. The maintenance medium can contain nutrients that sustain the embryogenic tissue and can also include hormones such as one or more auxins and/or cytokinins that promote cell division and growth of the embryogenic tissue. Typically, the concentration of hormones in the maintenance medium is lower than the concentration of hormones in the induction medium.

It is generally desirable, though not essential, to include maltose as the sole, or principal, metabolizable sugar source in the maintenance medium. Examples of useful maltose concentrations are within the range of from about 1% to about 2.5%. An example of a suitable maintenance medium is medium BM2 described in Table 1. Conifer embryogenic cells are typically transferred to fresh maintenance medium once per week. Although certain methods of induction and maintenance are described herein, any method for obtaining pre-cotyledonary somatic embryos can be used. Additional methods would be known to one skilled in the art.

Development.

In accordance with the described methods, a culture comprising conifer pre-cotyledonary somatic embryos is incubated in a development medium that promotes the development of cotyledonary embryos.

The development medium typically contains nutrients that sustain the somatic embryos. Suitable development media typically do not include growth-promoting hormones such as auxins and cytokinins. The osmolality of the development medium is in the range of from 300 mM/Kg to 500 mM/Kg. In some embodiments, the development medium has an osmolality of at least 350 mM/kg to 450 mM/kg or higher. An example of a suitable development medium is medium BM5 described in Table 1. Other suitable development media can also be used.

In one embodiment, the development medium comprises about 10% polyethylene glycol (PEG) to about 16% PEG. In one embodiment, the development medium comprises about 10% PEG to about 12% PEG (e.g., 10%, 11%, or 12% PEG).

Maltose can be included in the development medium as the principal or sole source of sugar for the somatic embryos. Useful maltose concentrations are within the range of from about 1% to about 2.5%.

The development medium can contain gellan gum. Gellan gum is a gelling agent marketed, for example, under the names GELRITE and PHYTAGEL. If gellan gum is included in the development medium, it is typically present at a concentration less than about 0.5%, typically at a concentration from about 0% to about 0.4%. The development medium is typically a solid medium, although it can be a liquid medium.

The development medium can contain an absorbent composition such as activated charcoal, such as the activated charcoal described herein in connection with induction medium.

The development medium can comprise sucrose and/or abscisic acid. The concentration of abscisic acid in the development medium can be between 0.5 mg/L and 500 mg/L. The concentration of abscisic acid in the development medium can be between 1 mg/L and 100 mg/L. The concentration of abscisic acid in the development medium can be between 5 mg/L and 20 mg/L.

The development medium can contain sucrose as the principal or sole source of metabolizable sugar. Useful sucrose concentrations are within the range of about 1% to about 6%.

The development medium can be liquid, solid, or semi-solid. The concentrations of osmoticants, such as PEG, or other osmoticants can be elevated in a liquid medium to produce the same osmolality as that of the corresponding solid medium. Typically, a solid development medium that is equivalent to a liquid development medium has an osmolality that is within about 50 mM/kg of the osmolality of the liquid development medium.

The development stage of somatic embryos can be divided into early stage, mid-stage, and late stage development. Early stage development includes histogenesis, or the formation of different tissues from undifferentiated cells. In particular, early stage development of an immature embryo includes root initial development, the beginning of root cap development, stele promeristem differentiation, and shoot apex formation.

Mid-stage development involves organ growth and the initiation of hypocotyl development and cotyledon development. Late stage development includes the completion of organ growth, the completion of hypocotyl and cotyledon development, and storage product deposition, resulting in an anatomically mature embryo.

The formation of one or more structures on one or more embryos (e.g., cotyledonary primordia, or cotyledons) can be determined by visual inspection or imaging analysis of the cultured embryos. Visual inspection or imaging analysis can be optionally conducted under 5-10× magnification.

In one aspect, the described methods comprise culturing conifer pre-cotyledonary somatic embryos in, or on, a development medium for a first incubation period sufficient in length for at least a portion of the pre-cotyledonary somatic embryos to develop into cotyledonary somatic embryos. The first incubation on the development media can be carried out at a temperature from 15° C. to 30° C., such as from 15° C. to 25° C., or such as from 20° C. to 22° C.

The first incubation period is sufficient in length for the formation of at least one of the following structures on a portion (e.g., at least one embryo, at least 10% of the embryos, at least 25%, at least 50%, more than 50%, or at least 75%) of the plurality of embryos in the first embryo culture: one or more embryos with cotyledonary primordia; one or more embryos with cotyledons; one or more embryos with 4+cotyledons; or one or more embryos with distinct cotyledons with hypocotyl and root regions present.

The length of the first incubation period can vary depending upon the particular genotype of the conifer pre-cotyledonary somatic embryo. In one embodiment, the first incubation period is from about 5 weeks to about 8 weeks, such as from about 6 weeks to about 7 weeks.

The described methods further comprise culturing the cotyledonary somatic embryos in, or on, the development medium for a second incubation period sufficient in length for at least a portion (e.g., at least one embryo, at least 10% of the embryos, at least 25%, at least 50%, more than 50%, or at least 75%) of the cotyledonary somatic embryos to reach anatomical maturity (i.e., possessing developed cotyledons and hypocotyl). In one embodiment, the cotyledonary somatic embryos are cultured in, or on, the development medium at a temperature from about 10° C. to about 15° C., such as 12° C.

In one embodiment, the second incubation period is from about 4 weeks to about 7 weeks, such as from about 5 weeks to about 6 weeks. In one embodiment, the first and second incubation periods together total about 12 weeks. For example, if the first incubation period were about 5 weeks, the second incubation period would be about 7 weeks. As another example, if the first incubation were about 6 weeks, the second incubation period would be about 6 weeks. As yet another example, if the first incubation period were about 7 weeks, the second incubation period would be about 5 weeks.

Stratification.

Stratification (moist chilling) is a treatment used for overcoming germination resistance in the seeds of many temperate species (Taylor & Waring, Plant, Cell, and Environment 2:165-171, 1979). Stratification can be carried out on development media comprising an osmolality of less than 150 mM/kg (such as BM5) up to 450 mM/kg.

Optionally, after incubating the somatic embryos on the development media for a second incubation period sufficient in length for at least a portion of the embryos to reach anatomical maturity, the embryos can then be subjected to stratification at a temperature of from 0° C. to 10° C. for an incubation period of at least one week up to 8 weeks, or from at least 2 months up to 6 months, to produce stratified cotyledonary somatic embryos that are stored prior to germination. The stratification period is typically carried out in the dark.

The initial osmolality of the development media at the start of the first incubation period is at least 300 mM/Kg, and is maintained at a level of at least 200 mM/kg during the stratification period. The osmolality level can be maintained by the addition of various osmoticants to the development media (e.g., PEG, various sugars, myo-inositol, or other osmoticants to increase osmolality), or by adjusting the volume of the development media the embryos are incubated in or on during development and stratification.

Typically, embryos form on the surface of a mass of embryogenic cells, such as an ESM. As described in more detail below, the cotyledonary embryos can be separated into individual (singulated) cotyledonary embryos before culturing them in, or on, the stratification medium, or they can be cultured as a mass of un-singulated embryos. Further, the cotyledonary embryos can be separated into individual (singulated) cotyledonary embryos before being subjected to a temperature of from 0° C. to 10° C. for a singulation period of at least one week to produce stratified cotyledonary somatic embryos, or they can be cultured as a mass of un-singulated embryos.

Singulation.

After stratification, the method optionally comprises singulating a plurality of individual embryos from the stratified cotyledonary somatic embryos. Any means of physically separating individual embryos from the stratified cotyledonary somatic embryos can be used to singulate the embryos. For example, in the context of an ESM culture, physical methods of separation can be used, such as washing away the ESM (e.g., spray singulation via pressure-controlled spray of aqueous liquid), vacuuming away the ESM, vibration, or picking the embryos from the ESM. Other non-limiting examples of useful singulation methods include filtering or sorting embryos based on a physical attribute such as size, shape, for example through a sieve, or based on other physical attributes such as surface roughness, hydrophobicity, density or mass.

The singulation step can include picking individual embryos based on one or more selection criteria. For example, visually evaluated screening criteria can be used by a skilled technician or a computerized imaging system to select embryos based on one or more morphological features including, but not limited to, the embryo's size, shape (e.g., axial symmetry), surface texture, color (e.g., no visible greening), absence of split hypocotyls, and no translucent cotyledons. Embryos can also be selected based on criteria relating to chemistry or external structure adsorption, reflectance, transmittance, or emission spectra through the use of near infrared spectroscopy (NIR), as described in U.S. Patent Application No. 2004/0072143, titled “Methods for Classification of Somatic Embryos,” the disclosure of which is incorporated herein by reference.

Desirable embryos can be individually picked (via a manual or automated process) out of the first embryo culture (e.g., such as an ESM), with any suitable instrument, such as tweezers. The embryo picking can be carried out manually or via an automated process, such as described in U.S. Pat. No. 7,530,197, titled “Automated System and Method for Harvesting and Multi-Stage Screening of Plant Embryos,” and in U.S. Pat. No. 8,621,943, titled “Method of Singulating Embryos,” the disclosures of which is incorporated herein by reference.

Germination.

Following stratification and singulation, anatomically mature embryos are germinated to form pine plants which can be grown into pine trees, if desired. Typically, cotyledonary embryos are subjected to a conditioning over water, or COW treatment, before germination. The cotyledonary embryos can also be inserted into manufactured seeds for subsequent germination. The cotyledonary embryos can be germinated, for example, on a solid germination medium, such as BM7 medium described in Table 1. Typically, all the steps of the described methods are conducted in the dark except for the germination step. During the germination step, cotyledonary embryos are kept in the dark for one week and in light for 5 weeks to stimulate germination. The germinated plants can be transferred to soil for further growth. For example, the germinated plants can be planted in soil in a greenhouse and allowed to grow before being transplanted to an outdoor site.

Although certain methods of stratification, singulation, and germination are described herein, any method known to one skilled in the art can be used.

In some embodiments of the method, late development of cotyledonary conifer somatic embryos in the cold at 12° C. to 15° C., after about 6 weeks of development, significantly improved the germination percentage as compared to conifer somatic embryos developed at 20° C. to 21° C. for 12 weeks.

Thus, in one aspect, a method for developing conifer cotyledonary somatic embryos is described. The method comprises the steps of (a) culturing conifer somatic cells in, or on, an induction medium to provide embryogenic cells; (b) culturing the embryogenic cells provided in step (a) in, or on, a maintenance medium to provide pre-cotyledonary somatic embryos; (c) culturing the pre-cotyledonary somatic embryos provided in step (b) in, or on, a development medium at a temperature from about 20° C. to about 22° C. for a first incubation period sufficient in length for at least a portion of the pre-cotyledonary somatic embryos to develop into cotyledonary somatic embryos; and (d) culturing the cotyledonary somatic embryos developed in step (c) in, or on, the development medium at a temperature from about 10° C. to about 15° C. for a second incubation period sufficient in length for at least a portion of the cotyledonary somatic embryos to reach anatomical maturity.

In one embodiment, the conifer pre-cotyledonary somatic embryos consist essentially of Loblolly pine pre-cotyledonary somatic embryos. In one embodiment, the conifer pre-cotyledonary somatic embryos consist essentially of Douglas-fir pre-cotyledonary somatic embryos.

In one embodiment, the development medium comprises about 10% PEG to about 16% PEG. In one embodiment, the development medium comprises about 10% PEG to about 12% PEG.

In one embodiment, the first incubation period is from about 5 weeks to about 8 weeks. In one embodiment, the second incubation period is from about 4 weeks to about 7 weeks. In one embodiment, the first and second incubation periods together total about 12 weeks.

In one embodiment, the cotyledonary somatic embryos of step (d) are cultured in, or on, the development medium at a temperature of 12° C.

The described methods can be used for producing a population of genetically-identical pine somatic embryos. The term “genetically-identical pine somatic embryos” as used herein refers to embryos that are derived from the same original plant. The term includes pine somatic embryos containing a small number of mutations that can occur during the development of somatic embryos. Any of the methods described herein can be used to produce populations of genetically-identical cotyledonary somatic pine embryos.

### Example 1

This example describes the late development of cotyledonary conifer somatic embryos in the cold at 12° C. to 15° C. compared to conifer somatic embryos developed at 20° C. to 21° C. for 12 weeks.

Cold late maturation at 12° C. after 6 weeks development was examined.

Development medium BM5 with 10% PEG was used to plate four genotypes of Loblolly pine conifer somatic embryos, specifically, genotypes A, B, C, and D. In the Treatment 1/control group, conifer somatic embryos were plated and developed at room temperature for 12 weeks. In the Treatment 8/cold late maturation group, conifer somatic embryos were plated and the plates were developed for 6 weeks at room temperature followed by 6 weeks at 12° C. Table 1 provides a comparison of the media used, including development medium BM5.

TABLE 1
Loblolly Pine Media Comparison

| | | BM 2 | BM 3 | | | | BM 7 |
|---|---|---|---|---|---|---|---|
| | BM 1 | “3x” | Liquid | BM 4 | BM 5 | BM 6 | Germ |
| | 1x Maint. | Maint. | Maint. | Rinse | Dev. | Cold | 185 |
| Salts (mg/L) | | | | | | | |
| NH4NO3 | 150 | 150 | 150 | 150 | 150 | 150 | 206.25 |
| KNO3 | 909.9 | 909.9 | 909.9 | 909.9 | 909.9 | 909.9 | 1170 |
| Ca(NO3)2•4H2O | 236.15 | 236.15 | 236.15 | 236.15 | 236.15 | 236.15 | |
| MgSO4•7H2O | 246.5 | 246.5 | 246.5 | 246.5 | 246.5 | 246.5 | 185 |
| Mg(NO3)2•6H2O | 256.5 | 256.5 | 256.5 | 256.5 | 256.5 | 256.5 | |
| MgCl2•6H2O | 50 | 50 | 50 | 50 | 50 | 50 | |
| KH2PO4 | 136 | 136 | 136 | 136 | 136 | 136 | 85 |
| CaCl2•2H2O | 50 | 50 | 50 | 50 | 50 | 50 | 220 |
| KI | 4.15 | 4.15 | 4.15 | 4.15 | 4.15 | 4.15 | 0.415 |
| H3BO3 | 15.5 | 15.5 | 15.5 | 15.5 | 15.5 | 15.5 | 3.1 |
| MnSO4•H2O | 10.5 | 10.5 | 10.5 | 10.5 | 10.5 | 10.5 | 8.45 |
| ZnSO4•7H2O | 14.4 | 14.4 | 14.4 | 14.4 | 14.4 | 14.4 | 4.3 |
| Na2MoO4•2H2O | 0.125 | 0.125 | 0.125 | 0.125 | 0.125 | 0.125 | 0.125 |
| CuSO4•5H2O | 0.125 | 0.125 | 0.125 | 0.125 | 0.125 | 0.125 | 0.0125 |
| CoCl2•6H2O | 0.125 | 0.125 | 0.125 | 0.125 | 0.125 | 0.125 | 0.0125 |
| FeSO4•7H2O | 27.87 | 27.87 | 27.87 | 27.87 | 27.87 | 27.87 | 13.93 |
| Na2EDTA | 37.26 | 37.26 | 37.26 | 37.26 | 37.26 | 37.26 | 18.63 |
| Vitamins/Amino | | | | | | | |
| Acids (mg/L) | | | | | | | |
| Nicotinic Acid | 0.5 | 0.5 | 0.5 | 0.5 | 0.5 | 0.5 | 0.5 |
| Pyridoxine HCl | 0.5 | 0.5 | 0.5 | 0.5 | 0.5 | 0.5 | 0.5 |
| Thiamine HCl | 1 | 1 | 1 | 1 | 1 | 1 | 1 |
| Glycine | 2 | 2 | 2 | 2 | 2 | 2 | 2 |
| L-proline | | | | 100 | 100 | 100 | |
| L-asparagine | | | | 100 | 100 | 100 | |
| L-arginine | | | | 50 | 50 | 50 | |
| L-alanine | | | | 20 | 20 | 20 | |
| L-serine | | | | 20 | 20 | 20 | |
| PEG 8000 mw | | | | | 100000 | | |
| Sugar/Agar mg/L | | | | | | | |
| Myo-Inositol | 200 | 200 | 200 | 100 | 100 | 100 | 100 |
| Casein hydrolysate | 500 | 500 | 500 | 500 | 500 | 500 | |
| L-glutamine | 1000 | 1000 | 1000 | 1000 | 1000 | 1000 | |
| Sucrose | | | | | | 25000 | 20000 |
| Maltose | 30000 | 30000 | 30000 | 25000 | 25000 | | |
| Glucose | | | | | 10000 | | |
| Gelzan | 1600 | 1600 | | | 2500 | 2500 | |
| TC Agar | | | | | | | 8000 |
| Activated carbon | | | | | 1000 | 1000 | 2500 |
| Hormones mg/L | | | | | | | |
| ABA | | | | | 25 | | |
| NAA | | | | | | | |
| 2,4-D | 1.1 | 3.0 | 1.1 | | | | |
| BAP | 0.1 | 0.25 | 0.1 | | | | |
| Kinetin | 0.1 | 0.25 | 0.1 | | | | |
| pH | 5.7 | 5.7 | 5.7 | 5.7 | 5.7 | 5.7 | 5.7 |

Rinse medium BM4, 2″×2″ Decotex nylon membrane, and plates of development medium BM5 (40 ml/plate) were used. Briefly, 30 ml of cells were transferred to a Cytostir stirred bioreactor flask. An equivalent amount of rinse medium BM4 was added to a stirred bioreactor flask to get the ratio 1:1 cells:rinse medium. One VPS unit was used for plating and frits were changed between genotypes. Six 2″×2″ pieces of 100 μm membrane were plated at a time with 1 ml of the cell/rinse medium mixture added onto each membrane. Plates were randomized when plating.

A cursory assessment was done from 3 to 8 weeks after plating to note any differences in the rate of development between treatments. Treatment 8 plates were assessed before being placed in the cold at 12° C., and plates were also assessed after 12 weeks of development. The final assessment included notes about embryo size, length, shape, color, texture, cotyledon formation, amount and condition of ESM, and any variation between or within plates.

Successful (and control) treatments were taken through to germination following post-development processes including stratification, spray separation, and transfer for germination. Specifically, all plates went to stratification in the cold on spent development medium at 12 weeks. Spray separation was used for COW; therefore, there was no embryo selection. Following spray separation, all objects on the S-frames were flipped over into Sigma germination boxes with 100 ml of germination medium BM7 using funnels and the flick and stick method. No imbibition was needed. All boxes spent one week in the dark before being transferred to a light room for a period of 5 weeks. Germination was assessed at 6 weeks.

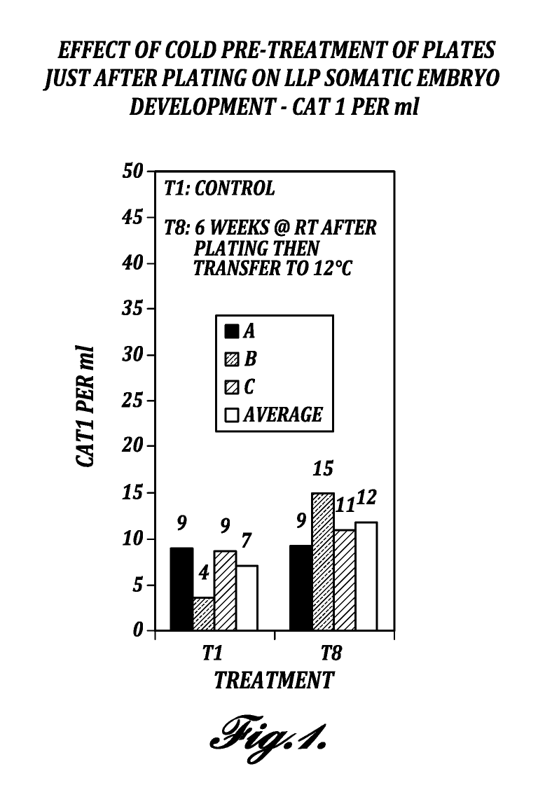

Genotype D exhibited low category 1 germination and was excluded from subsequent analysis. FIG. 1 shows category 1 germinants per ml. Because spray separation was used, only the number of germinants of category 1 per germination box were counted.

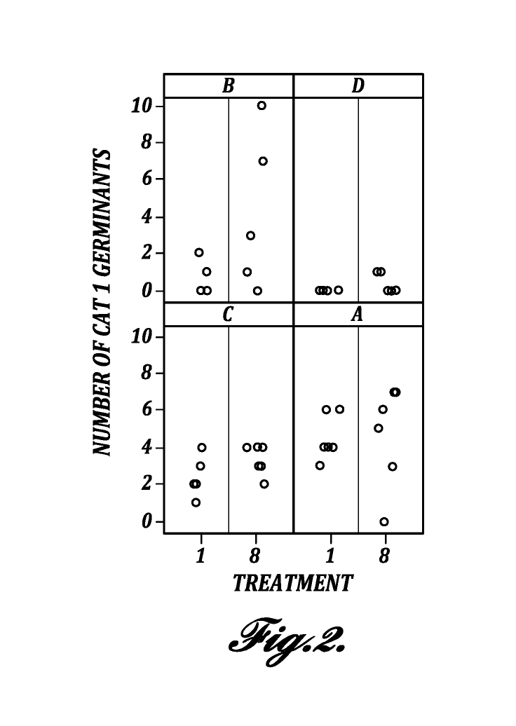

Statistical analysis on the number of category 1 germinants was performed. The responses were analyzed using a generalized linear model with the Poisson distribution and log link. Treatment means and confidence intervals were transformed back to the natural scale for all responses. FIG. 2 shows the number of category 1 germinants percentage for all the S-frames. The X-axis shows the treatments (Treatment 1/Control or Treatment 8) and the panels show the genotypes. Genotype D was excluded from the analysis because of a lack of category 1 germinants.

There is strong evidence of a treatment effect (p-value<0.0001). Treatment LS-means and pairwise comparisons are given in Table 2. Table 2 provides the mean number of category 1 germinants. L90 and U90 are the lower and upper 90% confidence limits, respectively, for each mean. The column “Test at a=0.10” summarizes test results comparing combined means. Means with the same symbol are not statistically different at α=0.10.

TABLE 2

| | | Test at | | |
|---|---|---|---|---|
| Treatment | Mean | α = 0.10 | L90 | U90 |
| 1 | 2.3 | B | 1.8 | 3.1 |
| 8 | 4.1 | A | 3.3 | 5.1 |

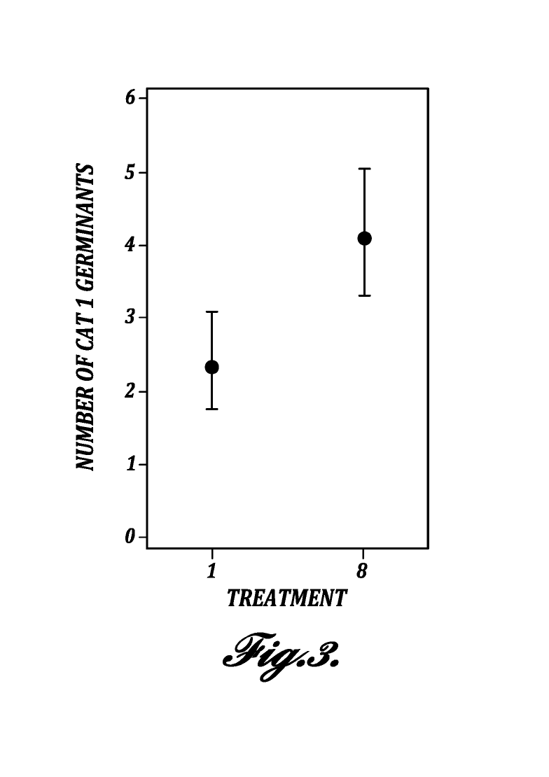

The means and confidence intervals are plotted in FIG. 3. Error bars are 90% confidence limits.

FIG. 4 shows category 1+2 germinants per ml. Because spray separation was used, only the number of germinants of category 1 per germination box were counted.

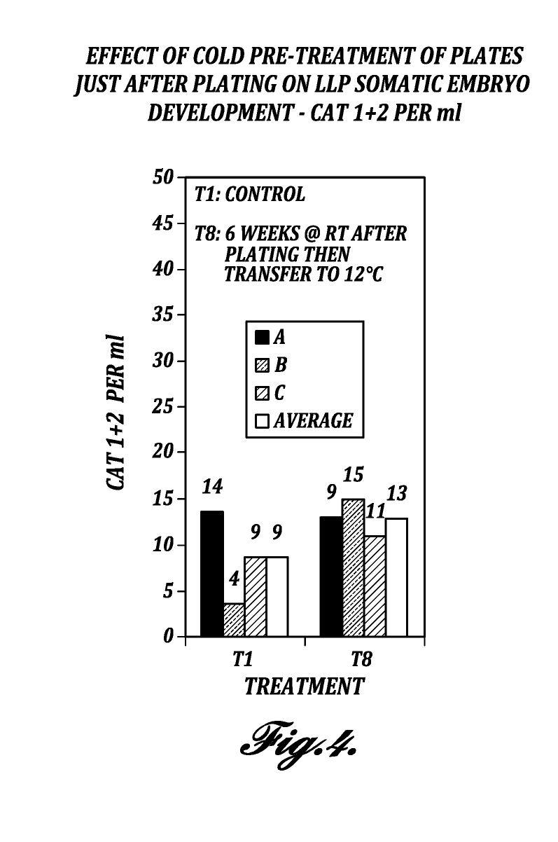

FIG. 5 shows the number of category 1+2 germinants percentage for all the S-frames in the experiment. The X-axis shows the treatments and the panels show the genotypes. All genotypes were included in this analysis.

There is strong evidence of a treatment effect (p-value=0.015). Treatment LS-means and pairwise comparisons are given in Table 3. L90 and U90 are the lower and upper 90% confidence limits, respectively, for each mean. The column “Test at α=0.10” summarizes test results comparing combined means. Means with the same symbol are not statistically different at α=0.10.

TABLE 3

| | | Test at | | |
|---|---|---|---|---|
| Treatment | Mean | α = 0.10 | L90 | U90 |
| 1 | 2.9 | B | 2.3 | 3.6 |
| 8 | 3.9 | A | 3.2 | 4.8 |

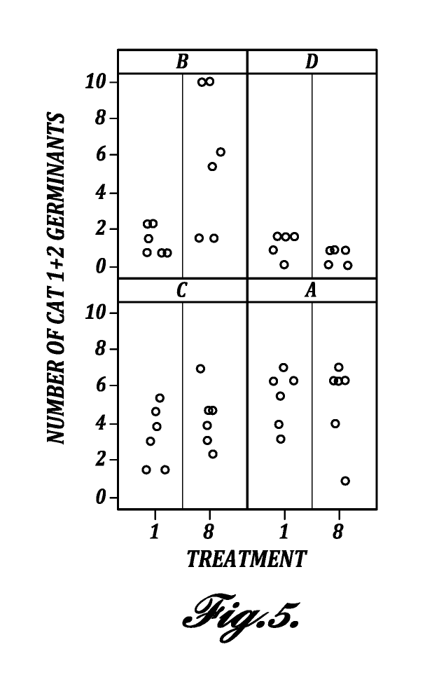

The means and confidence intervals are plotted in FIG. 6. Error bars are 90% confidence limits.

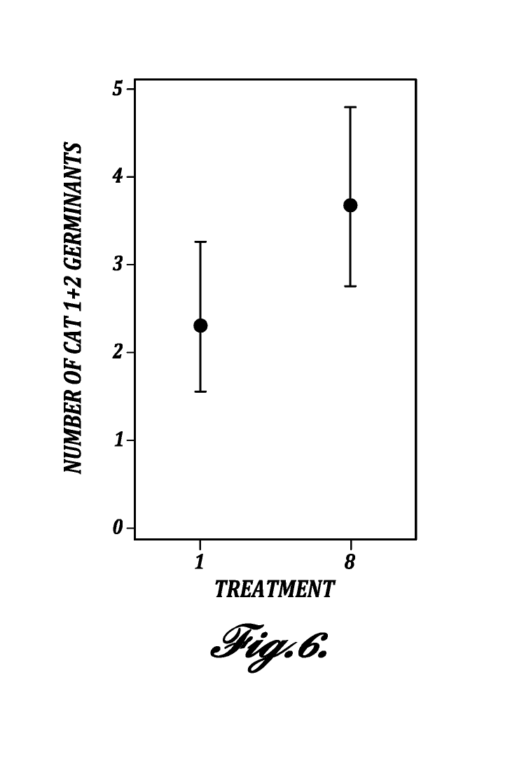

FIG. 7 shows the percent increase in category 1 germinants when Treatment 8 was compared to the control group for three genotypes: A, B, and C. As shown in FIG. 7, there was approximately a 300% increase in category 1 germinants for genotype B compared to genotypes A and C over the control group.

FIG. 8 shows the percent increase in category 1+2 germinants when Treatment 8 was compared to the control group for three genotypes: A, B, and C. As shown in FIG. 8, there was approximately a 300% increase in category 1 germinants for genotype B compared to genotypes A and C over the control group.

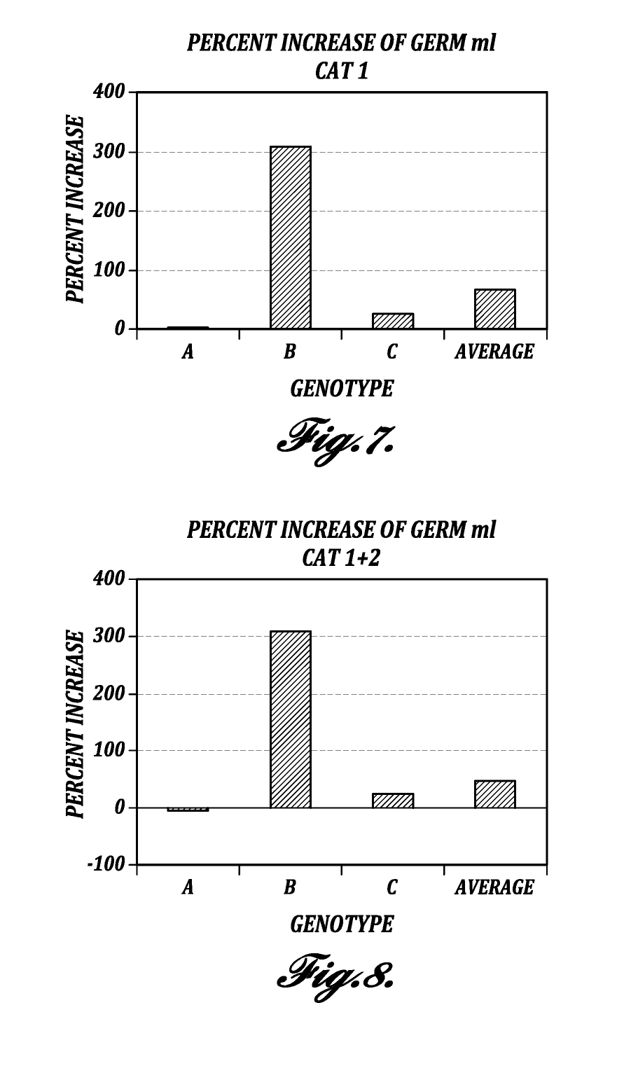

FIG. 9 shows root length for genotypes A, B, and C.

The results demonstrate how the late development of cotyledonary conifer somatic embryos in the cold at 12° C. to 15° C., following about 6 weeks of development at room temperature, significantly and unexpectedly improved the germination percentage when compared to conifer somatic embryos developed at 20° C. to 21° C. for 12 weeks.

### Example 2

This example describes the late development of cotyledonary conifer somatic embryos in the cold at 12° C. to 15° C. compared to conifer somatic embryos developed at 20° C. to 21° C. for 12 weeks.

Cold late maturation of cotyledonary somatic embryos at 12° C. was examined. Development medium BM5 with 10% PEG was used. There were five ⅓ Cambro boxes per treatment. Two boxes were sacrificed for osmolality measurements at 5 and 8 weeks development. Three boxes continued for 12 weeks development. The initial osmolarity measurement of the medium was taken using a VAPRO 5520 (Wescor Inc.) vapor pressure osmometer. Treatments of Loblolly pine genotypes E, F, and D are shown in Table 4.

TABLE 4

| | | | | | | Average |
|---|---|---|---|---|---|---|
| Treatment | Medium | PEG | Maltose | Glucose | Sucrose | osmos |
| Control | BM5 | 10% | 2.5% | 1% | 0 | 347 |
| Treatment 7 | BM5 | 10% | 2.5% | 1% | 0 | 347 |

Development medium BM5 with 10% PEG was used with 400 ml per ⅓ Cambro box. Cambro boxes were taken to the lab for plating. Cells were plated onto D-frames with one D-frame per box. The standard treatment for plating was to plate 25% culture to 76% rinse of the scheduled genotypes onto a D-frame using an automated plating method and an automated method of separation and singulation carried out in a laboratory clean room.

Embryo development into full cotyledon stage was carried out on development medium BM5 with 10% PEG for all genotypes. The Treatment 1/control Cambro box was developed for 12 weeks at room temperature. The Treatment 7/cold late maturation Cambro box was developed at room temperature until the conifer somatic embryos reached cotyledonary stage, a period of about 6 weeks, then moved to 12° C. for a few more weeks until 12 weeks total time of development was reached.

Post development, the embryos were moved to stratification medium BM6 after 12 weeks on development medium. For stratification, 200 ml of stratification medium BM6 was used per ⅓ Cambro box. Spray separation and singulation using an automated method was carried out in a clean room. Three D-frames per treatment were combined and spray separated. Somatic embryos were then transferred for germination using a Sigma germination box and 100 ml of germination medium BM7. The selection criteria for embryos were as follows. Following 12 weeks of development and after a week in a full COW box with 500 ml sterile water, 25 embryos were transferred from an S-frame to a Sigma germination box.

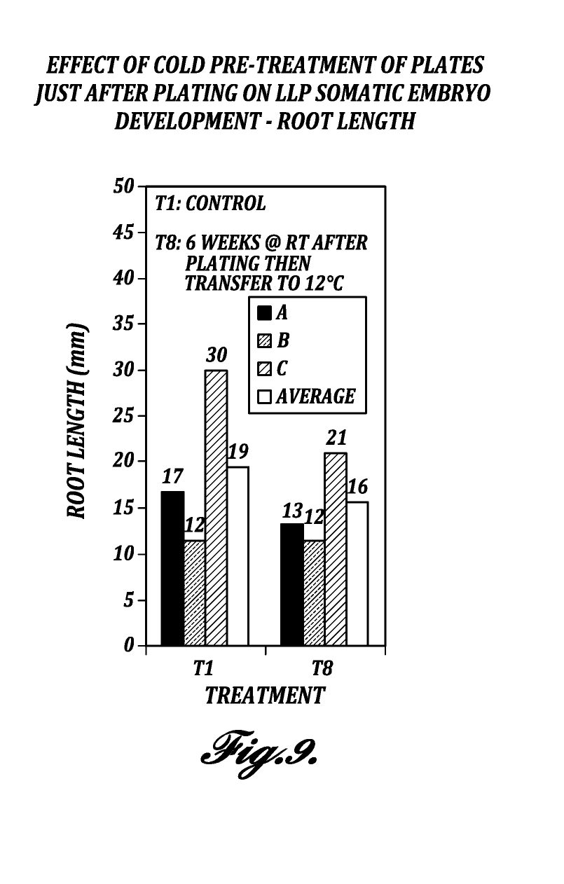

Depending on the formation of cotyledonary embryos in each genotype, the move time to the cold temperature (12° C.) varied from 6 weeks to 7 weeks. Genotypes E and F were moved to the cold temperature after 6 weeks of development while genotype D was moved to the cold temperature after 7 weeks of development. Following one week in the dark and 5 weeks in the light, the embryos were assessed for germination. The percentage of category 1 germinants for both treatments is shown in FIG. 10.

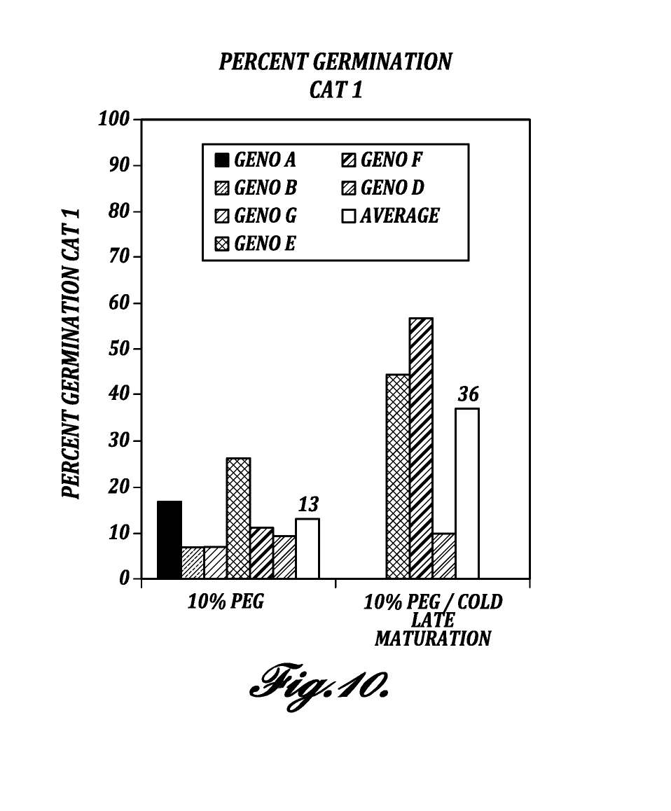

FIG. 11 shows the germination percentage for category 1 germinants for the three genotypes used in Example 2. The X-axis shows the treatment (Treatment 1 or Treatment 7) and the panels show the genotypes (F, E, or D).

There is strong evidence of a treatment effect (p-value<0.0001). Treatment LS-means and pairwise comparisons are given in Table 5. L90 and U90 are the lower and upper 90% confidence limits, respectively, for each mean. The column “Test at α=0.10” summarizes test results comparing combined means. Means with the same symbol are not statistically different at α=0.10.

TABLE 5

| | | Test at | | |
|---|---|---|---|---|
| Treatment | Mean | α = 0.10 | L90 | U90 |
| 1 | 0.12 | B | 0.09 | 0.17 |
| 7 | 0.33 | A | 0.28 | 0.40 |

The LS-means and confidence intervals for the data in FIG. 11 are plotted in FIG. 12. FIG. 2 provides the LS-means for category 1 germination for all treatments. Error bars are 90% confidence limits.

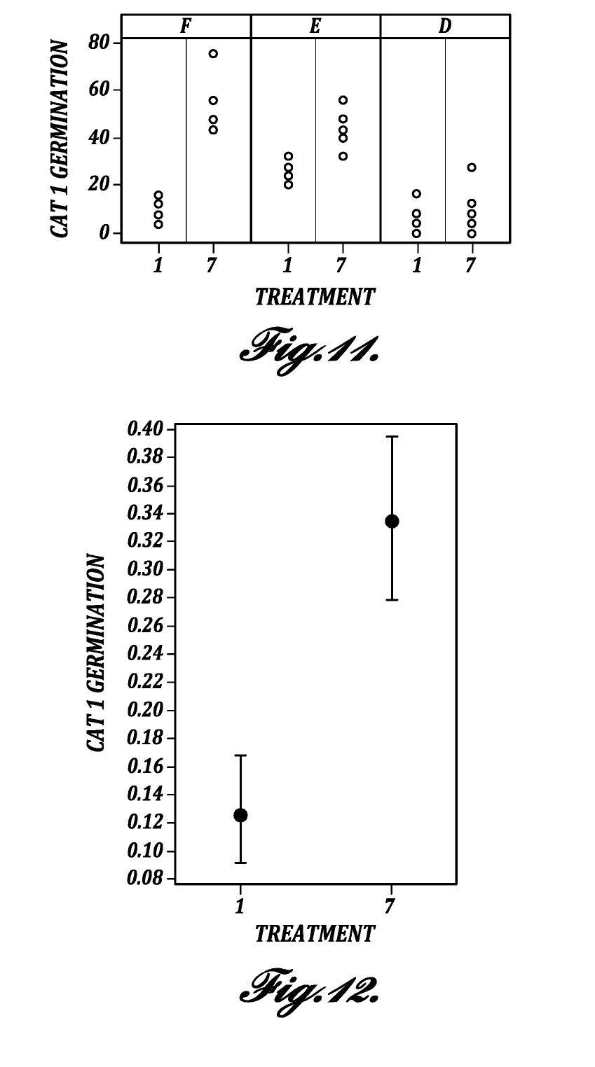

Root Length. FIG. 13 shows the root length for each category 1 germinant by genotype. The X-axis shows the treatments and the panels show the genotypes.

There is no evidence of a treatment effect (p-value=0.19). Treatment LS-means and pairwise comparisons are given in Table 6. L90 and U90 are the lower and upper 90% confidence limits, respectively, for each mean.

TABLE 6

| Treatment | Mean | L90 | U90 |
|---|---|---|---|
| 1 | 14.4 | 11.8 | 16.9 |
| 7 | 17.2 | 14.6 | 19.8 |

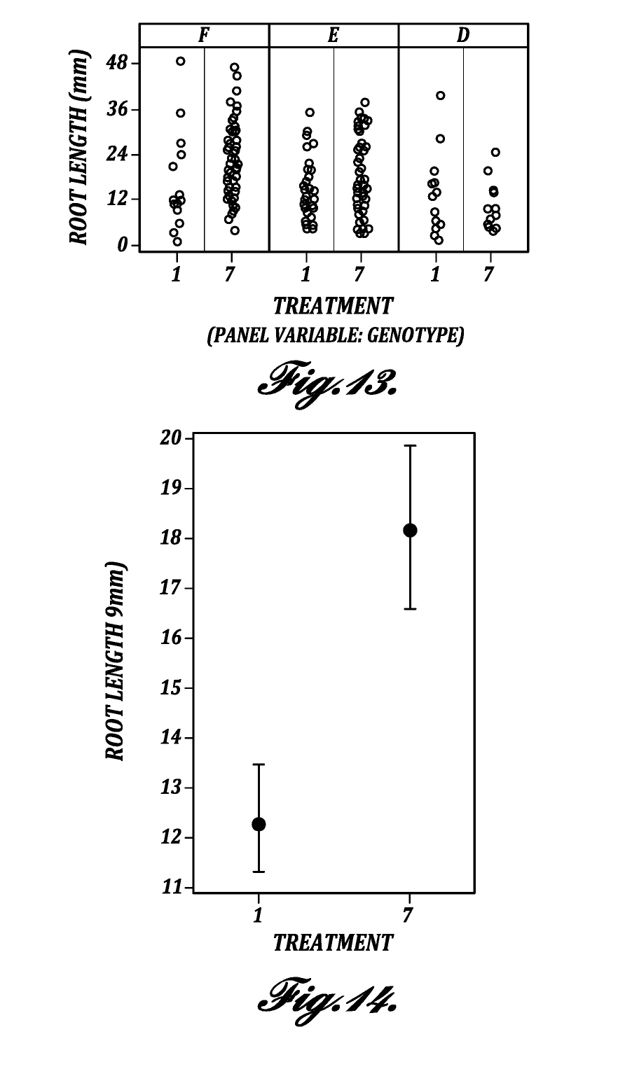

The means and confidence intervals for the data shown in FIG. 13 are plotted in FIG. 14.

Hypocotyl Length.

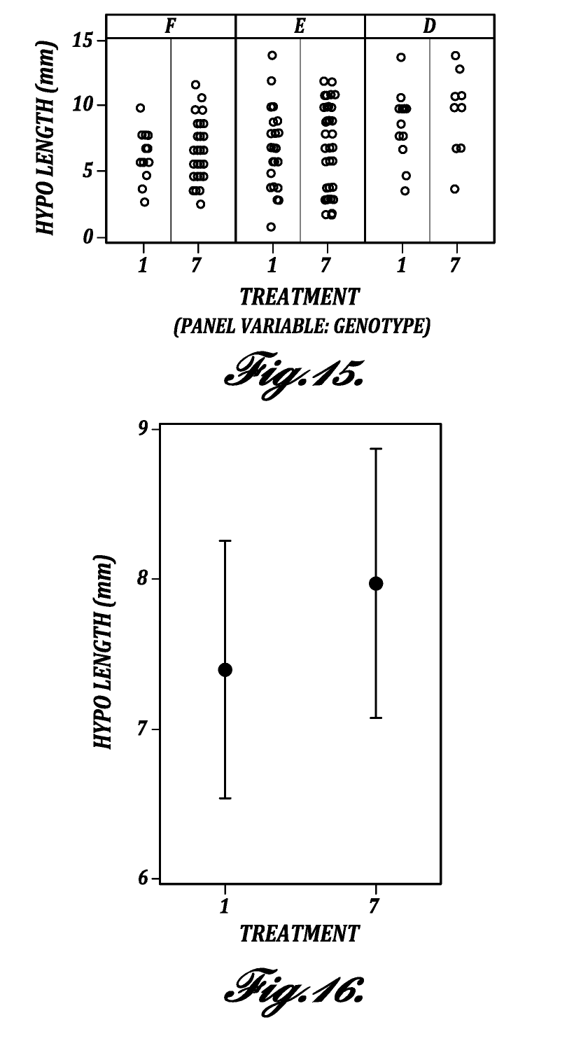

FIG. 15 shows the hypocotyl length for each category 1 germinant by genotype. The X-axis shows the treatment and the panels show the genotypes.

There is no evidence of a treatment effect (p-value=0.44). Treatment LS-means and pairwise comparisons are given in Table 7.

TABLE 7

| Treatment | Mean | L90 | U90 |
|---|---|---|---|
| 1 | 7.4 | 6.5 | 8.3 |
| 7 | 8.0 | 7.1 | 8.9 |

The LS-means and confidence intervals for hypocotyl length for both treatments are plotted in FIG. 16. L90 and U90 are the lower and upper 90% confidence limits, respectively, for each mean.

Epicotyl Length.

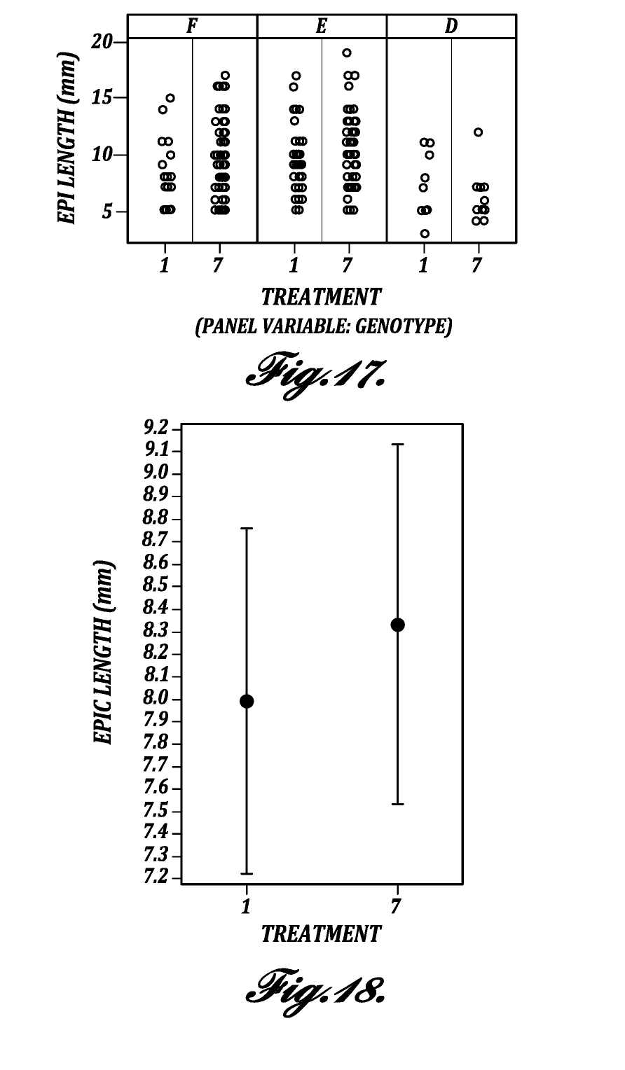

FIG. 17 shows the epicotyl length for each category 1 germinant by genotype. The X-axis shows the treatment and the panels show the genotypes.

There is no evidence of a treatment effect (p-value=0.61). Treatment LS-means and pairwise comparisons are given in Table 8.

TABLE 8

| Treatment | Mean | L90 | U90 |
|---|---|---|---|
| 1 | 8.0 | 7.2 | 8.8 |
| 7 | 8.3 | 7.5 | 9.1 |

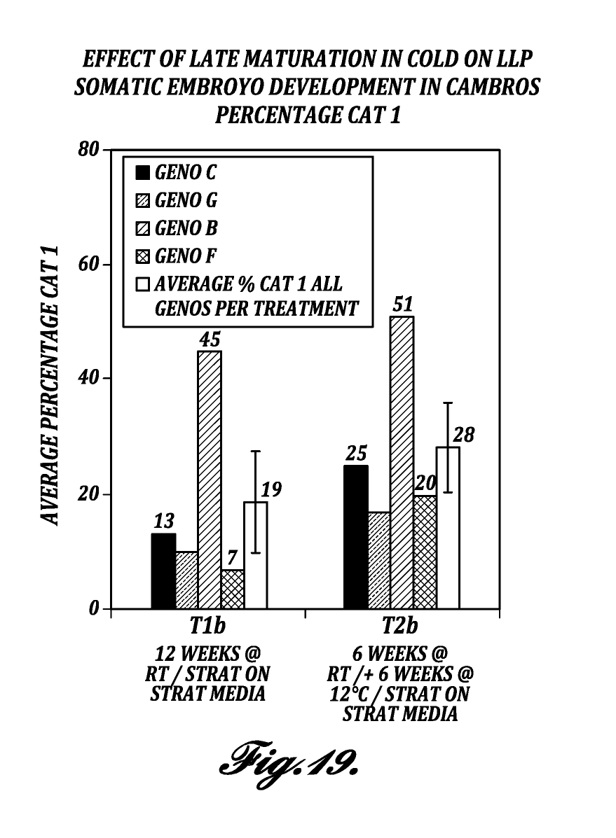

The LS-means and confidence intervals for epicotyl length for Treatment 1 and 7 are plotted in FIG. 18. L90 and U90 are the lower and upper 90% confidence limits, respectively, for each mean.

The results show how the late development of cotyledonary conifer somatic embryos in the cold at 12° C. to 15° C., following about 6 weeks of development at room temperature, significantly improved the germination percentage as compared to conifer somatic embryos developed at 20° C. to 21° C. for 12 weeks. Development boxes kept at 12° C. to 15° C. for late maturation were found to have less ESM and improved embryo quality as compared to the control. Germination of cold late maturation treatment was statistically improved over the control. There were no significant differences in root, hypocotyl and epicotyl lengths between the control and cold late maturation treatment.

### Example 3

This example describes the late development of cotyledonary conifer somatic embryos in the cold at 12° C. to 15° C. compared to conifer somatic embryos developed at 20° C. to 21° C. for 12 weeks.

Late embryo development was slowed down after 6 weeks development at 20° C. to 21° C. by lowering the temperature to 12° C. Genotypes C, G, B and F were used, with three ½ Cambro boxes per genotype. One extra ½ Cambro box was used for dry weight, assays, Redox, Metabolon, non-sterile picture and osmolarity measurements at 6 weeks of development at room temperature.

Development medium BM5 with 12% PEG was used. All boxes of each genotype were developed at room temperature for 6 weeks before moving to the cold at 12° C. for all genotypes. A 6-week window of time for moving the somatic embryos to 12° C. was used, with a total development time of 12 weeks, as shown below in Table 9.

TABLE 9

| | | | Total | |
|---|---|---|---|---|
| | | | development | |
| Treatment | @RT | Move to 12° C. | time | Stratification |
| Treatment 1b | 12 wks | N/A | 12 wks | Strat on |
| | | | | stratification |
| | | | | media BM6 |
| Treatment 2b | 6 wks | 6 wks | 12 wks | Strat on |
| | | | | stratification |
| | | | | media BM6 |

Because there were 12 weeks of development, Treatments 1 and 2 were blocked and sprayed separated at the same time. Successful (and control) treatments were taken through to germination following the processes below.

Post development, stratification occurred after 12 weeks of development. Spray separation and singulation occurred in the lab using an automated method. S-frames were placed into a 1/1 COW box. Spray separation embryos were kept in COW from 7 to 10 days. Automated flip and stick was carried out using an AFS (5/13) box type ⅓ Cambro with a clear lid and 400 ml of germination medium BM7. After one week in the dark, germination boxes were placed in a lighted room for 5 weeks. Germination assessment was carried out at 6 weeks. Surprisingly, germination frequency was improved in the cold late maturation treatment group compared to the control group.

FIG. 19 shows the effect of late maturation in the cold on Loblolly pine somatic embryo development. The X-axis shows the treatment type (Treatment 1 or Treatment 2) for each of the 4 genotypes (C, G, B, F), and the Y-axis shows the average percentage of category 1 germinants. As shown in FIG. 19, late maturation at 12° C. to 15° C. unexpectedly improved germination for each genotype compared to control.

While the preferred embodiment has been illustrated and described, it will be appreciated that various changes can be made therein without departing from the spirit and scope of described embodiment.

The embodiments in which an exclusive property or privilege is claimed are defined as follows:

1. A method for developing conifer cotyledonary somatic embryos comprising the steps of:
(a) culturing conifer pre-cotyledonary somatic embryos in, or on, a development medium at a temperature from about 20° C. to about 22° C. for a first incubation period sufficient in length for at least a portion of the pre-cotyledonary somatic embryos to develop into cotyledonary somatic embryos; and
(b) culturing the cotyledonary somatic embryos developed in step (a) in, or on, the development medium at a temperature from about 10° C. to about 15° C. for a second incubation period sufficient in length for at least a portion of the cotyledonary somatic embryos to reach anatomical maturity.

2. The method of claim 1, wherein the conifer pre-cotyledonary somatic embryos consist essentially of Loblolly pine pre-cotyledonary somatic embryos.

3. The method of claim 1, wherein the conifer pre-cotyledonary somatic embryos consist essentially of Douglas-fir pre-cotyledonary somatic embryos.

4. The method of claim 1, wherein the development medium comprises about 10% PEG to about 16% PEG.

5. The method of claim 4, wherein the development medium comprises about 10% PEG to about 12% PEG.

6. The method of claim 1, wherein the first incubation period is from about 5 weeks to about 8 weeks.

7. The method of claim 6, wherein the first incubation period is from about 6 weeks to about 7 weeks.

8. The method of claim 1, wherein the second incubation period is from about 4 weeks to about 7 weeks.

9. The method of claim 8, wherein the second incubation period is from about 5 weeks to about 6 weeks.

10. The method of claim 1, wherein the first and second incubation periods together total about 12 weeks.

11. The method of claim 1, wherein in step (b) the cotyledonary somatic embryos are cultured in, or on, the development medium at a temperature of 12° C.

12. A method for developing conifer cotyledonary somatic embryos comprising the steps of:
(a) culturing conifer somatic cells in, or on, an induction medium to provide embryogenic cells;
(b) culturing the embryogenic cells provided in step (a) in, or on, a maintenance medium to provide pre-cotyledonary somatic embryos;
(c) culturing the pre-cotyledonary somatic embryos provided in step (b) in, or on, a development medium at a temperature from about 20° C. to about 22° C. for a first incubation period sufficient in length for at least a portion of the pre-cotyledonary somatic embryos to develop into cotyledonary somatic embryos; and
(d) culturing the cotyledonary somatic embryos developed in step (c) in, or on, the development medium at a temperature from about 10° C. to about 15° C. for a second incubation period sufficient in length for at least a portion of the cotyledonary somatic embryos to reach anatomical maturity.

13. The method of claim 12, wherein the conifer pre-cotyledonary somatic embryos consist essentially of Loblolly pine pre-cotyledonary somatic embryos.

14. The method of claim 1, wherein the conifer pre-cotyledonary somatic embryos consist essentially of Douglas-fir pre-cotyledonary somatic embryos.

15. The method of claim 12, wherein the development medium comprises about 10% PEG to about 16% PEG.

16. The method of claim 12, wherein the development medium comprises about 10% PEG to about 12% PEG.

17. The method of claim 12, wherein the first incubation period is from about 5 weeks to about 8 weeks.

18. The method of claim 12, wherein the second incubation period is from about 4 weeks to about 7 weeks.

19. The method of claim 12, wherein the first and second incubation periods together total about 12 weeks.

20. The method of claim 12, wherein in step (d) the cotyledonary somatic embryos are cultured in, or on, the development medium at a temperature of 12° C.
